(19) United States
(12) Patent Application Publication
(10) Pub. No.: US 2026/0206934 A1
(43) Pub. Date: Jul. 23, 2026

(54) Systems and Methods of Altering Hair Texture

(71) Applicant: Tanisha Knox, Los Angeles, CA (US)

(72) Inventor: Tanisha Knox, Los Angeles, CA (US)

(21) Appl. No.: 19/570,146

(22) Filed: Mar. 17, 2026

Related U.S. Application Data
(63) Continuation-in-part of application No. 18/042,217, filed on Aug. 23, 2021.
(60) Provisional application No. 63/069,231, filed on Aug. 24, 2020.

(51) Int. Cl. A45D 7/04; A61Q 5/04; A61Q 5/12

(52) U.S. Cl. CPC A45D 7/04; A61Q 5/04; A61Q 5/12

(57) ABSTRACT

Systems and methods for altering hair texture discloses the system comprising a porosity mixture; a protective mixture; a texture altering mixture comprising a low concentration alkaline agent and an integrated conditioning matrix for active keratin disulfide bond loosening to achieve predictable, partial, graduated curl modification while preserving curl structure of a curly, coily, or wavy curl pattern of hair; a neutralizing shampoo mixture; a moisturizing shampoo mixture; and a conditioning mixture.

### (B) CROSS-REFERENCE TO RELATED APPLICATIONS

This application is a continuation-in-art of pending U.S. patent application Ser. No. 18/042,217, filed on Feb. 19, 2023, which claims priority to and benefit of national stage application, filed under 35 U.S.C. § 371, of International Patent Application No. PCT/US2021/47099, filed on Aug. 23, 2021, which claims priority to and the benefit of U.S. Provisional Patent Application No. 63/069,231, filed on Aug. 24, 2020, each of which are incorporated herein by reference in its entirety.

### (C) FEDERALLY SPONSORED RESEARCH AND DEVELOPMENT

Not applicable.

### (D) BACKGROUND OF THE INVENTION

### 1. Field of the Invention

Embodiments herein relate generally to systems and methods for altering hair texture and, more specifically, to systems and methods for altering the curl pattern of hair using a low concentrate alkaline agent with an integrated conditioning base for reforming a curl pattern without straightening the hair.

### 2. Description of Arguably Related Art Including Information Disclosed For 37 CFR 1.97 and 1.98.

Problems exist with conventional hair texture altering mixtures, compositions, systems, and methods because many of these systems use caustic chemicals, nonorganic components or ingredients, and result in permanent straightening of a natural curl pattern to a straight curl pattern of hair. These systems may be referred to as chemical straighteners. Examples of chemical straighteners include, but are not limited to, hair relaxers or hair texturizers. In general, a hair relaxer (often called lanthionization) often includes a straighten fixing agent, for example sodium hydroxide having a concentration of at least 1.5% by weight in combination with other nonorganic ingredients, which is applied to at least the roots of the hair strand for up to about 20 minutes before rinsing with water. Other sodium hydroxide/lye relaxers or chemical straighteners contain about 1.5% to 2.5% by weight sodium hydroxide relative to the total composition. “Stronger” chemical straighteners may have 3.0% to 3.5% by weight sodium hydroxide to achieve a desired straightening effect. After washing the chemical straightener from the hair, the processed strands are permanently straightened from an initial or previous curly, coily, or wavy curl pattern of hair.

Hair textures come in a variety of curl patterns from no curl pattern-essentially straight hair—to wavy, to curly, to super curly curl patterns of hair. References to “super curly curl patterns” further includes coily to super coily curl patterns. A curl pattern can be altered in different ways by straightening, reforming, loosening the disulfide bonds of a curl pattern of hair. To straighten a curl pattern, the hair may be stretched of the curl, coil, or wave through mechanical altering or manipulation or through chemical altering or manipulation. Problems attributed to chemical straighteners, such as relaxers and texturizers, include caustic chemicals that may burn the scalp, overprocessing the hair resulting in damage to the hair follicles whenever the relaxer or texturizer is applied for too long without rinsing and neutralizing with neutralizing shampoo.

Some conventional chemical straighteners use a fixing agent that is capable of forming or causing crosslinks with proteinaceous hair keratin. These compositions may include, but are not limited to, dimethyl ketone, butanone, nbutanol, heptanone, ethanol, t-butanol, and others. One or more these compositions may be toxic and/or carcinogenic even in low concentrations. These fixing agents may also use small organic molecules selected for the ability to induce or participate in keratin crosslinking reactions. Many chemical straighteners use high proportions of the fixing agent, for example 1% to 45% by weight, which teaches or suggests the significance of these keratin-reactive agents for application of its chemical straighteners to structurally alter hair. Conditioning treatment of the hair is often used as a secondary or post-process step in the relaxing process.

U.S. Published Patent Application No. US2009/0165812 filed by Resnick et al. “Resnick” discloses a hair texture altering system, the hair texturing system comprising: a texture altering mixture. Further provided are one or more hair straightening system comprising (a) the hair straightening formulations set forth above; (b) one or more shampoos, and (c) one or more conditioners; and a protective mixture. The systems include a skin protectant. For the scalp protectant, coat and gently rub the scalp with a thin film of cellulose sulfate gel using a small hand-held applicator or fingers. The cellulose sulfate is applied as a gel (alkaline pH at 9-10) to the scalp concentrations ranging from 0.6%-5% with a preferred concentration of 2-3%. It further includes a porosity mixture, wherein the porosity mixture is formulated to prepare the hair for receiving the texture altering mixture. The systems include a hair relaxer. This product will swell and open the cuticle and make it semi-porous due to the breakage of the disulfide bond; a shampoo. The systems include a hair relaxer (e.g., shampoo). A shampoo that is slightly basic (pH 7-8) and containing citric acid (buffered with sodium citrate) can be used to relax the hair; and a conditioning mixture, wherein the conditioning mixture is configured to improve a condition of the hair. The systems include a hair fixative. The hair fixative preparation contains hydrolyzed keratin and epigallocatechin gallate as the other main active ingredients. It has been found that the use of particular natural compound in predetermined proportions achieves a straightening effect while simultaneously conditioning the hair.

U.S. Published Patent Application No. US2004/0110650 filed by Siddiqui et al. (“Siddiqui”) discloses a hair texture altering system, the hair texture system comprising: a texture altering mixture (shampoo and conditioner); a shampoo mixture, wherein the formulation is included in separate hair care products (such as a shampoo and a conditioner); and a conditioning mixture, wherein the conditioning mixture is configured to improve a condition of the hair.

U.S. Pat. No. 5,060,680 issued to Akhtar discloses a method of altering hair texture comprising the steps of: applying a texture altering mixture to hair. Auxiliary amounts of the texturing and strengthening agent can be applied to the virgin outgrowth before applying the hair straightener. Employing a shampoo is usually preferred for purposes of cleansing and removing from the hair residual hair straightener.

A need exists for systems and methods for altering hair texture that have a porosity mixture; a protective mixture; a texture altering mixture comprising a low concentration alkaline agent and an integrated conditioning matrix for active keratin disulfide bond loosening to achieve predictable, partial, graduated curl modification while preserving curl structure of a curly, coily, or wavy curl pattern of hair; a neutralizing shampoo mixture; a moisturizing shampoo mixture; and a conditioning mixture.

A need exists for systems and methods for altering hair texture without the use of free of parabens, sulfates, silicones, synthetic fragrance, mineral oil, petrolatum, and synthetic petrochemicals.

A need exists for systems and methods for altering hair texture by reforming the curl pattern rather than straightening the curl pattern.

A need exists for systems and methods for altering hair texture by reforming the curl pattern rather than straightening the curl pattern by adding moisture to the hair using one or more natural oils.

A need exists for systems and methods for altering hair texture by loosening, reshaping, and reforming the curl pattern of hair to a looser curl or coil rather than straightening the hair.

None of the patent references disclose the invention.

### (E) Summary of the Invention

It is understood that the present disclosure is not limited in its application to the details of construction or to the arrangement of the components described in the following illustrated drawing or in the detailed description.

Provided herein are systems and methods for altering hair texture and, more specifically, systems and methods for altering the curl pattern of hair without straightening the hair in a manner that reduces the use of harmful and/or synthetic materials while using natural and/or plant-based or plant-derived ingredients that improve the condition of the hair and that reduce the possibility of damage to the hair. A hair texture altering system of an embodiment of the present disclosure comprises a texture altering mixture, wherein the texture altering mixture is formulated to reform and reshape, but not straighten, a curl pattern of hair, for example, coily hair, super coily hair, curly hair, super curly hair, or wavy hair. The texture altering system may also add moisture to the hair using one or more natural oils, which may include about 0.10% by weight Simmondsia chinensis (jojoba) seed oil and/or about 0.10% by weight Helianthus annuus (sunflower) seed oil. The texture altering mixture has a low concentrate alkaline agent. The low concentrate alkaline agent is preferably sodium hydroxide (NaOH). The texture altering mixture may include about 1.35% by weight sodium hydroxide, about 1.48% by weight of sodium hydroxide, or about 1.35% to 1.75% by weight sodium hydroxide. It should be noted that the amount of concentration of sodium hydroxide used with the texture altering mixture is not strong enough to damage or to convert or change the hair from a coily, curly, or wavy curl pattern to straight hair. Instead, an effect on the altered curl pattern of hair may be similar to tension being applied to the curl pattern to lengthen and loosen the curl pattern. The hair would still be considered “naturally” coily, curly, or wavy, at least in appearance. The texture altering mixture may also be free of parabens, sulfates, silicones, synthetic fragrance, mineral oil, petrolatum, and synthetic petrochemicals.

The texture altering system of an embodiment of the present disclosure also includes a protective mixture, wherein the protective mixture is formulated to form a barrier between the texture altering mixture and a scalp portion of a head located at the root of the hair. The protective mixture may include one or more natural oils, for example, Simmondsia chinensis (jojoba) seed oil. The protective mixture may include a base ingredient, where the base ingredient includes at least one or more natural oils, and where at least one of such natural oils may include about 55.0% by weight Helianthus annuus (sunflower) seed oil. The protective mixture may further include Curcuma longa (turmeric) root extract. The protective mixture may have a consistency of a gel-like oil, and may be free of parabens, sulfates, silicones, synthetic fragrance, mineral oil, petrolatum, and synthetic petrochemicals.

The texture altering system of an embodiment of the present disclosure also includes a porosity mixture, wherein the porosity mixture is formulated to prepare the hair for receiving the texture altering mixture by coating the hair with proteins and/or amino acids for even distribution and absorption of the texture altering mixture on the hair.

The texture altering system of an embodiment of the present disclosure further includes a neutralizing shampoo mixture, where the neutralizing shampoo mixture is formulated to return the hair to its normal pH level and to add moisture to the hair. The neutralizing shampoo mixture may include 0.10% by weight of aloe barbadensis leaf juice to add moisture to the hair. The neutralizing shampoo may be free of parabens, sulfates, silicones, synthetic fragrance, mineral oil, petrolatum, and synthetic petrochemicals.

The texture altering system of an embodiment of the present disclosure also includes a moisturizing shampoo mixture, where the moisturizing shampoo mixture is formulated to moisturize the hair using, for example, about 0.10% by weight of aloe barbadensis leaf juice to moisturize the hair.

The texture altering system of an embodiment of the present disclosure further includes a conditioning mixture, where the conditioning mixture is configured to improve a condition of the hair by moisturizing the hair using one or more natural oils, and where at least one of the one or more natural oils is selected from a group consisting of about 1.50% by weight of Simmondsia chinensis (jojoba) seed oil and about 1.50% by weight of persea gratissima (avocado) oil, guar hydroxypropyltrimonium chloride, and Oryza sativa (rice) bran extract. The conditioning mixture may also strengthen the hair by using one or more proteins, and where at least one of the one or more proteins is selected from a group consisting of hydrolyzed wheat protein, hydrolyzed soy protein, or hydrolyzed corn protein. The conditioning mixture may also be free of parabens, sulfates, silicones, synthetic fragrance, mineral oil, petrolatum, and synthetic petrochemicals.

The present disclosure further includes a method of altering hair texture comprising the steps of: applying a protective mixture, wherein the protective mixture is formulated to create a barrier between the texture altering mixture and a scalp portion of a head located at the root of the hair; applying a porosity mixture to the hair, wherein the porosity mixture is formulated to prepare the hair for application of the texture altering mixture; applying a texture altering mixture to hair, wherein the texture altering mixture is formulated to reform, but not straighten, a curl pattern of the hair; applying a neutralizing shampoo mixture to the hair, wherein the neutralizing shampoo mixture is formulated to return the hair to its normal pH level and to add moisture to the hair; applying a moisturizing shampoo mixture to the hair, wherein the moisturizing shampoo mixture is formulated to moisturize the hair; and applying a conditioning mixture, wherein the conditioning mixture is configured to improve the condition of the hair, and wherein improving the condition of the hair comprises moisturizing and strengthening the hair. The texture altering mixture of the method may include about 1.35% by weight sodium hydroxide or about 1.48% by weight sodium hydroxide. Effective curl reformation was achieved with about 1.35% by weight sodium hydroxide.

The texture altering system employs a low concentration alkaline agent or pH adjuster within an integrated conditioning matrix that enables real-time modulation of hair fiber response during processing of the hair. Implementing the disclosed systems and methods of the invention yielded unexpected results. Application of the method allows for reproducible, adjustable outcomes across multiple hair types that correlate to processing time and approach used with the system as opposed to a conventional chemical straighteners system employing a stepwise approach or a single application of a chemical straightener. The observed preservation of tensile strength and elasticity at effective curl-loosening levels would not be reasonably expected by one of ordinary skill in the art based on traditional chemical straightener teachings, which often associate sodium hydroxide processing with cumulative fiber degradation and cross-linking chemistry.

It is an objection of this invention to provide systems and methods for altering hair texture comprising a porosity mixture; a protective mixture; a texture altering mixture comprising a low concentration alkaline agent and an integrated conditioning matrix for active keratin disulfide bond loosening to achieve predictable, partial, graduated curl modification while preserving curl structure of a coily, curly, or wavy curl pattern of hair; a neutralizing shampoo mixture; a moisturizing shampoo mixture; and a conditioning mixture.

It is an object of this invention to provide systems and methods for altering hair texture without the use of free of parabens, sulfates, silicones, synthetic fragrance, mineral oil, petrolatum, and synthetic petrochemicals.

It is yet another object of this invention to provide systems and methods for altering hair texture by reforming the curl pattern rather than straightening the curl pattern.

It is an object of the invention to provide systems and methods for altering hair texture by reforming the curl pattern rather than straightening the curl pattern by add moisture to the hair using one or more natural oils.

These and other aspects, objects, embodiments, and advantages of the invention will become apparent from the accompanying drawing and the following detailed description of the preferred embodiments of the invention.

### (F) BRIEF DESCRIPTION OF THE DRAWING

The invention may be more readily described by reference to the accompanying drawing figures and the following description of the drawing figures. The reference numbers apply to each embodiment of the invention. In the drawing:

FIG. 1 illustrates an exemplary hair texture altering system consistent with an embodiment of the present disclosure.

FIG. 2 illustrates an exemplary method for altering hair texture consistent with an embodiment of the present disclosure.

### (G) DETAILED DESCRIPTION OF THE INVENTION

A detailed description of the embodiments of the present disclosure is provided below. While several embodiments are described, the disclosure is not limited to any one embodiment, but instead encompasses numerous alternatives, modifications, and equivalents. In addition, while numerous specific details are set forth in the following description to provide a thorough understanding of the embodiments disclosed herein, some embodiments can be practiced without some or all of these details. Moreover, for clarity, certain technical material that is known in the related art has not been described in detail to avoid unnecessarily obscuring the disclosure.

The description may use the terms “embodiment,” or “embodiments,” which may each refer to one or more of the same or different embodiments. The terms “comprising,” “including,” “having,” and the like, as used with respect to embodiments, are synonymous, and are generally intended as “open” terms, for example, the term “includes” should be interpreted as “includes but is not limited to,” the term “including” should be interpreted as “including but not limited to,” and the term “having” should be interpreted as “having at least.”

Regarding the use of any plural and/or singular terms herein, those of skill in the relevant art can translate from the plural to the singular and/or from the singular to the plural as is appropriate to the context and/or application. The various singular and/or plural permutations may be expressly set forth herein for the sake of clarity.

As used in this description, the term “a” or “an” means “at least one” or “one or more.” The singular forms “a,” “an,” and “the” include plural references unless the context clearly dictates otherwise. Any reference to “or” herein is intended to encompass “and/or” unless otherwise stated. The term “about” or “approximately” refers to a range of values within plus or minus 10% of the specified number.

Unless otherwise defined, all technical terms used herein have the same meaning as commonly understood by one of ordinary skill in the art to which this disclosure belongs.

The embodiments of the disclosure may be understood by reference to the drawing figures, wherein like parts may be designated by like numerals. The components of the disclosed embodiments, as generally described and illustrated in the figures herein, could be arranged and designed in a variety of different configurations. Thus, the following detailed description of the embodiments of the disclosure is not intended to limit the scope of the disclosure, as claimed, but is merely representative of possible embodiments of the disclosure. In addition, the steps of any method disclosed herein may include variations, or even variations sequentially, and the step may be repeated, unless otherwise specified.

Generally, human hair texture is determined by shape. As hair grows out from the scalp, it assumes the shape of the follicle. Hair textures may come in a variety of curl patterns and porosities. Examples of curl patterns are essentially straight strands with little to no perceptible curls, waves, or coils; wavy strands; curls having loose or tight S-shaped or spiral or corkscrew type formations; coils having tighter S-shaped formation as compared with S-shaped curls; and coils having perceptively undefined coil formations. A person may naturally have one or more curl patterns growing from the scalp. The flatter the cross-section of a strand of hair, the curlier the hair. The more circular the cross-section of a strand of hair, the straighter the hair. The more oval-shaped the cross-section of a strand of hair, the wavier the hair.

Additionally, for everyone, each strand of hair is formed of proteins called keratin. There are three types of chemical bonds that attract the keratin proteins to each other and are responsible for the strength and elasticity of human hair-namely, hydrogen, salt, and disulfide bonds. Disulfide bonds, in particular, add strength to the keratin protein. Thus, disulfide bonds must be altered to change the shape of the hair.

Situations and preferences exists for systems and methods for altering one's hair texture and curl pattern. Some options with existing systems and methods alter the curl pattern, whether permanently, semi-permanently, or reversibly using mixtures and formulations, or mechanical or physical alteration of the curl pattern. These approaches often entirely or substantially remove and transform the curl pattern of hair from coily, curly, or wavy to a straight or nearly-straight with little to no curl or wave in the curl pattern of hair. For a mechanical or physical approach, styling tools may be used to manipulate the curl pattern, for example, curling irons or flat irons that use heat to temporarily change the curl pattern, or wash and go styles used to temporarily define the curl pattern.

Another common approach to reshaping curlier hair involves the use of chemical straighteners such as hair relaxers, hair texturizers, and other similar products. A hair relaxer is essentially a mixture or formulation applied to the root of the hair and/or initially along the length of the hair strand or shaft to chemically and permanently alter a curl pattern to a straight texture, non-curl or coil pattern. These products typically contain harsh and/or noxious chemicals such as formaldehyde, glutaraldehyde and the like, or high concentrations of sodium hydroxide (about 2.0% to 2.5% by weight), which are designed to break the disulfide bonds in the hair to straighten the hair. This process, however, may leave the hair straightened, weak, brittle, and prone to breakage. Such products may even burn the skin, cause permanent damage to the scalp, and lead to hair loss. Moreover, such products may contain petroleum-based ingredients, mineral oil, animal-derived ingredients, and synthetic ingredients, each of which may be harmful.

Another distinguishing feature between the present disclosure and a chemical straightener is that with a conventional chemical straightener, the disulfide bonds are broken to such an extent that the original or modified curl, coil, or wavy curl pattern is permanently altered. In this situation, the hair does not revert back to its original or modified curl, coil, or wavy curl pattern either upon time or upon one or more subsequent washing sessions. Another distinguishing feature between a chemical straightener and the present disclosure is the length of time the product is applied to the hair before rinsing. Duration of the chemical straightener and the initial hair texture may impact the final result and the amount of straightening impacted on the curl pattern. On subsequent applications or “retouches,” the chemical straightener is only applied to the recent natural curl pattern hair growth or “new growth” near the line of demarcation between previously processed or relaxed hair and the new growth or roots of natural curl pattern between the scalp and the processed hair. The chemical straightener is preferably not applied to the previously treated hair strands on a subsequent day of application due to the high risk of the hair strands being damaged or suffering from breakage, sometimes referred to as overprocessing the hair.

A hair texturizer is a system and method for semi-permanent processing of a curl pattern to straighten the hair, often applied for less time than a chemical straightener. As with the permanent bond-breaking chemical straightener, the texturizer is initially applied to the roots and along the entire hair strands. Subsequent applications are only applied to the new growth of hair and not the remainder of the hair strand. Texturized hair also typically does not revert back to its natural curl pattern of coils, curls, or waves either over time or upon exposure to increased moisture, for example, washing the hair.

Mechanical manipulation is a temporary hair styling option for changing the curl pattern. However, a problem is reversion of the hair due to subsequent moisture. Damage can also be caused from improper use or overuse of heat on the hair, which permanently alters the hair strands, preventing reversion to the natural curl pattern. Another challenge with mechanical manipulation is inconsistent results.

To solve these problems, various embodiments of the present disclosure herein include systems and methods for altering hair texture comprising a porosity mixture; a protective mixture; a texture altering mixture comprising a low concentration alkaline agent and an integrated conditioning matrix for active keratin disulfide bond loosening to achieve predictable, partial, graduated curl modification while preserving curl structure of a curly, coily, or wavy curl pattern of hair; a neutralizing shampoo mixture; a moisturizing shampoo mixture; and a conditioning mixture. More particularly, a system and methods are disclosed that, among other things, modifies the disulfide bonds of the hair only to the extent to loosen, reform, or reshape the curl pattern of the hair without straightening the hair by using oils, other natural ingredients, and lower concentrations of sodium hydroxide. Additionally, such embodiments reduce the use of harsh and/or synthetic chemicals like those found in most chemical straighteners or similar products. With this system and method, a person having a super curly or curly curl pattern may utilize this system to keep the coils, curls, or waves yet treat the hair with a formulation designed to create semi-permanent looser, larger, or smaller coils, curls, or wavy curl pattern without permanently straightening or otherwise damaging the hair. Furthermore, the system and method allows for a more consistent curl pattern. For example, the texture altering mixture may be applied to one section of hair by selectively using a concentration of sodium hydroxide for a desired duration, while the texturing altering mixture may be applied to a different section of hair by selectively using a different concentration of sodium hydroxide for a desired duration before rinsing. In this example, a person having multiple different curl patterns of hair on their head can essentially “treat” the different curl patterns to yield the look of a more unified curl pattern result.

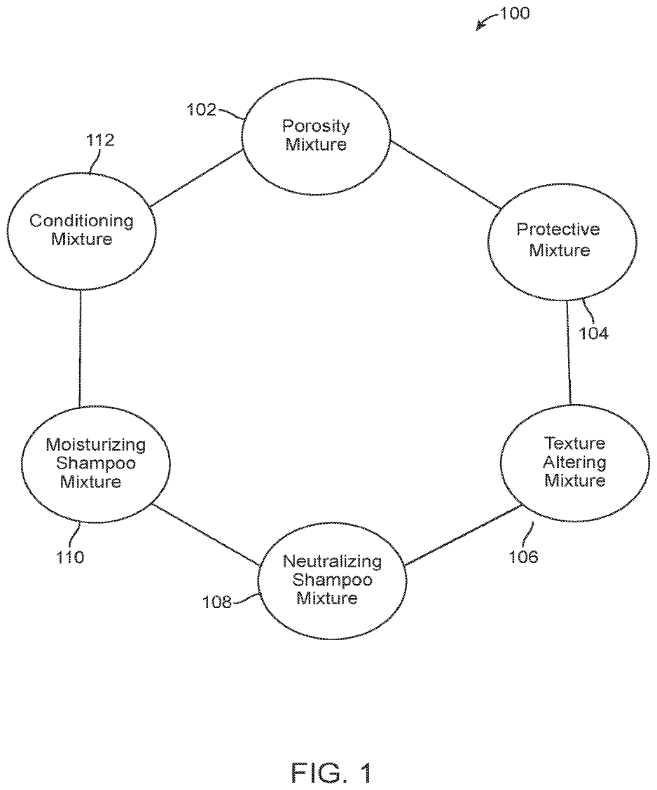

In accordance with the various embodiments herein, an exemplary hair texture altering system 100 is shown in FIG. 1. In one embodiment, the texture altering system 100 may comprise (include or have) a porosity mixture 102, a protective mixture 104, a texture altering mixture 106, a neutralizing shampoo mixture 108, a moisturizing shampoo mixture 110, and a conditioning mixture 112. In another embodiment, the texture altering system may comprise the porosity mixture 102, the protective mixture 104, the texture altering mixture 106, the neutralizing shampoo mixture, 108, the moisturizing shampoo mixture 110, and the conditioning mixture 112, wherein the texture altering mixture 106 may be applied to the hair for a duration up to about 60 minutes before rinsing, have a low concentration of an alkaline agent, namely sodium hydroxide, for use as a pH adjuster for the mixture rather than for breaking the keratin disulfide bond to straighten the hair, and may be applied in subsequent applications selectively from the roots to the ends of the hair strand or to only the roots or new growth of the hair. The system essentially reshapes and reforms curly, coily, or wavy hair into a looser form while maintaining the natural look of the curl pattern. Do to the formulation of at least the texture altering mixture of the system, the curl pattern of hair or hair strands are not straightened and are only stretched. The texture altering mixture may be applied to the hair shaft and/or new growth or roots of the hair on subsequent applications. When applied to the previously modified hair shaft on subsequent applications, the curl pattern of hair may further loosen or reform while never actually straightening.

In one embodiment of a hair texture altering system, the hair texture altering system comprising (including or having):
a. a texture altering mixture comprising a low concentration alkaline agent and an integrated conditioning agent for active keratin disulfide bond loosening and reforming a curl pattern of hair for graduated curl pattern modification while preserving curl structure for subsequent curl pattern reversion and without straightening the hair;
b. a porosity mixture, wherein the porosity mixture is formulated to prepare the hair for receiving the texture altering mixture;
c. a protective mixture, wherein the protective mixture is formulated to form a barrier between the texture altering mixture and a scalp portion of a head located at a root of the hair;
d. a neutralizing shampoo mixture, wherein the neutralizing shampoo mixture is formulated to return the hair to its normal pH level and to add moisture to the hair;
e. a moisturizing shampoo mixture, wherein the moisturizing shampoo mixture is formulated to moisturize the hair, wherein improving a condition of the hair comprises moisturizing the hair using one or more natural oils; and
f. a conditioning mixture, wherein the conditioning mixture is configured to improve the condition of the hair.

Consistent with embodiments herein, each of the mixtures 102-112 of the hair texture altering system 100 may be formulated to be free of parabens, sulfates, silicones, synthetic fragrance, harmful ingredients, as well as scalp irritating ingredients that may cause a dry and/or flaxy scalp. Each of the mixtures 102-112 of the hair texture altering system 100 may also be formulated using a majority natural, plant, and/or plant-derived ingredients, including natural and essential oils, and the like. Additionally, each of the mixtures 102-112 may comprise a PETA Certification, for example, a vegan and cruelty-free certification, and be free from animal testing.

The hair texture altering system contains no reactive molecules capable of performing or inducing keratin crosslinking. Here, the composition is approximately 94% plant-derived and uses non-toxic, biocompatible ingredients. The compositions are specifically formulated to avoid harsh volatile organics, ketones, aldehydes, alcohols, or analogous reactive agents. The mixtures, particularly the texture altering mixture, are chemically incapable of participating in the crosslinking reactions that define the process in conventional relaxers, texturizers, or other chemical-based hair altering compositions. It should be noted that the minor, trace levels of sodium hydroxide does not induce or initiate crosslinking on its own and does not, by itself, perform structural protein-bond modification of hair strands. Instead, as disclosed, the sodium hydroxide is used strictly as a pH adjuster. It cannot create or promote covalent bonds with keratin because the mixtures do not include any compatible, reactive organic molecules or ingredients for reactive chemistry. Accordingly, sodium hydroxide at the preferred concentrations is unsuitable as a fixing agent and cannot be used in the mixture to straighten the curl pattern of hair through breaking disulfide bonds as new structure.

Here, the ingredients or composition in the texture altering mixture modify hair texture through a gentle, non-reactive, plant-derived mechanism that involves temporarily softening, hydrating, and mechanical restructuring of the curl pattern of hair. The texture altering mixture does not create new disulfide or covalent bonds. It does not break or reform keratin linkages. It also does not rely on reactive solvents or ketones. Instead, the hair altering system produces a modified texture alteration without structural keratin damage.

The system and method were tested to evaluate the performance, predictability, and impact on hair fiber integrity when used pursuant to the ascribed and/or preferred methods of use. Testing was conducted to assess the degree of curl pattern reformation without straightening the hair during and after application of the texture altering mixture; to observe changes to hair fiber integrity; to evaluate the consistency of results across multiple hair types and processing ranges; and to compare outcomes of using the system with the texture altering mixture against conventional chemical straightener systems and structural curl-relaxation approaches. Here, the system is designed and configured for incomplete, graduated bond modification, reshaping, or reforming of the hair. The unexpected results from testing the system and method yielded effective curl loosening in addition to preserving and maintaining fiber integrity for multiple curl patterns or hair types, from wavy to curly to super curly, at a low concentration of the alkaline agent used in the texture altering mixture. Furthermore, the texture altering mixture of the system integrates conditioning and any chemical reactions to yield results in contrast to conventional chemical straightener systems that generally have separate conditioning and chemical reaction functionality.

Table 1 is a chart of the testing parameters for the system.

TABLE 1
Testing Parameters for Hair Texture Altering System

| Parameter | Description |
|---|---|
| Product | Texture Altering System |
| Use Classification | Professional-use only |
| Testing Methods | Strand testing, live model application, repeat |
| | application observation |
| Hair Types Evaluated | (wavy to curly to coily) |
| Hair Condition | Virgin, previously colored, previously |
| | bleached, and previously |
| | chemically treated (case-dependent) |
| Processing Control | Time-based, visually monitored |

Using the testing parameters, observation was made to the curl pattern loosening after application of the system 100 and is represented in Table 2 as curl loosening percentages.

TABLE 2
Curl Loosening Percentages and Comparison

| Hair Type/Baseline Curl Pattern | Observed Curl Loosening Range |
|---|---|
| Loose wave | 10-25% elongation |
| Defined curls | 20-40% elongation |
| Tight curls | 25-45% elongation |
| Coils | 20-40% elongation |
| Tight coils | 15-35% elongation |

Table 3 reflects a chart reporting hair fiber integrity after application of the system, with scoring using a 1 to 5 scale. The scoring definition is defined as 5=no observable degradation; 4=minor, manageable change; 3=moderate change requiring conditioning support; 2=significant compromise; and 1=severe compromise.

TABLE 3
Hair Fiber Integrity Scores

| | Average | |
|---|---|---|
| Metric Evaluated | Score | Notes |
| Tensile Strength | 4-5 | Maintained when protocol followed |
| Elasticity | 4 | Slight reduction in higher curl patterns |
| Surface Smoothness | 4-5 | Improved with Texture Altering Care |
| | | Creme use |
| Breakage Incidence | 4-5 | Minimal under controlled application |
| Overall Fiber Integrity | 4-5 | No widespread degradation observed |

The system may be implemented using the disclosed parameters of concentration, processing time, and conditioning integration to produce consistent, reproducible results. Strand testing demonstrated a high correlation between preliminary assessment and final outcome.

The texture altering system provides for increased customization of the curl pattern by allowing for customized implementation of the system at a particular step, for example, using a certain concentration of sodium hydroxide or selecting a particular natural oil. Another customization option allows for a single pass of application of the mixtures. In contrast, other structural curl-related systems require a fixed process. Here, conditioning integration is integral to the hair altering system. In contrast, conditional integration for conventional chemical straightening systems may apply only in the post-processing portion of the system. The texture altering system preserves curl retention. Unlike some chemical straightening systems, for example relaxers, where results may be inconsistent and unpredictable, the texture altering system has a higher rate of predictability and consistency.

The texture altering system is further distinguishable from conventional hair relaxers in that this system uses a low concentration alkaline agent (NaOH) and one or more conditioning agents to achieve partial, graduated curl modification while preserving curl structure. The system integrates conditioning concurrently with active bond loosening of the hair to deliberately moderate reaction kinetics to produce non-binary outcomes of incremental curl loosening ranges of approximately 15%-45% rather than maximum bond disruption from a relaxer.

### Porosity

In one embodiment, the porosity mixture 102 of the system 100 may prepare the hair for application of the texture altering mixture 106 by coating the hair with protein and amino acids or the like to fill in any pores, such as open spaces, that may exist in the hair cuticle. This coating allows for even distribution and absorption of the texture altering mixture 106 to prevent overly porous hair from absorbing the texture altering mixture 106 unevenly and/or faster than desired.

The porosity mixture 102 may be applied to the shaft and the ends of the hair while dry. No rinsing is required. In one embodiment, the porosity mixture 102 may be formulated as a liquid that can be sprayed on the hair; however, other formulations of the porosity mixture 102 are possible without departing from embodiments of the present invention. During the application process of the system, the porosity mixture 102 is applied before the texture altering mixture 106. The porosity mixture is part of the pre-treatment mixture.

In one embodiment, the porosity mixture 102 may comprise the ingredients shown in Table 4. All percentages in Table 4 are by weight and approximate for the entire composition, making up 100% as shown in Table 4.

TABLE 4
Porosity Mixture Example

| INCI Name | Purpose | Percentage |
|---|---|---|
| Water | | 76.78 |
| Propanediol | Humectant | 10.00 |
| Tetrasodium Glutamate | Chelator | 0.20 |
| Diacetate | | |
| Sodium Citrate | pH Adjuster | 0.10 |
| Citric Acid | pH Adjuster | 0.22 |
| Water (and) Wheat | Active-Hair | 6.00 |
| Amino Acids (and) Soy | Conditioning | |
| Amino Acids (and) | | |
| Arginine HCl (and) | | |
| Serine (and) Threonine | | |
| Water (and) Hydrolyzed | Active-Hair | 3.00 |
| Wheat Protein (and) | Conditioning | |
| Hydrolyzed Soy Protein | | |
| (and) Hydrolyzed Corn | | |
| Protein | | |
| Polyquaternium-73 | Active-Hair | 2.00 |
| | Conditioning | |
| Cetrimonium Chloride | Active-Hair | 0.20 |
| | Conditioning | |
| Sodium Benzoate (and) | Preservative | 1.00 |
| Potassium Sorbate (and) | | |
| Water | | |
| Panthenol | Active - Hair | 0.50 |
| | Conditioning | |
| | | 100% |

### Protection

Referring still to FIG. 1, the protective mixture 104 of the system 100 may protect the scalp during application of the texture altering mixture 106 by creating a barrier between the skin and the texture altering mixture 106. In one embodiment, by applying the protective mixture 104 directly to the scalp as a pre-treatment prior to applying the texture altering mixture 106 to the hair, the possibility of skin irritation is reduced.

In one embodiment, the protective mixture 104 may comprise various natural and/or essential oil ingredients, which give the protective mixture 104 a gel-like oil or “gel-oil” consistency. By using natural and/or essential oils to formulate the protective mixture 104, potentially harmful ingredients such as mineral oils, petroleum-based ingredients, synthetic ingredients, and the like, can be avoided.

For example, consistent with embodiments of the present disclosure, a base, namely, a principle ingredient, of the protective mixture 104 may be formulated by using one or more natural and/or essential oils. In one embodiment, as shown in Table 5 below, a base of about 55% by weight Helianthus annuus seed oil (sunflower oil) may be used, although other natural oils such as coconut oil, and the like, are possible. In another example, one or more essential oils may be used to formulate the protective mixture 104 for fragrance. For example, in one embodiment, as shown in Table 5, the following essential oils may be combined to form a fragrance of the protective mixture 104: thuja occidentalis leaf (white cedar) oil, Ormenis multicaulis (chamomile) oil, eugenia caryophyllus (clove) leaf oil, Cymbopogon Martini oil, pelargonium graveolens flower (geranium) oil, and Fusanus spicatus wood (sandal wood) oil. However, other essential oils may be used alone or in combination to form a fragrance of the protective mixture 104. Using essential oil(s) to form the protective mixture 104 fragrance alleviates the use of synthetic or other chemical ingredients that are typically used to create fragrance in hair products. The protective mixture is part of the pre-treatment mixture.

In one embodiment, the protective mixture 104 may comprise the ingredients shown in Table 5 below. All percentages in Table 5 are by weight and approximate for the entire composition, making up 100% as shown in Table 5.

TABLE 5
Protective Mixture Example

| INCI Name | Purpose | Percentage |
|---|---|---|
| Helianthus Annuus (Sunflower) Seed Oil | Emollient | 55.00 |
| Tribehenin | Thickener | 6.00 |
| Glyceryl Myristate (and) Glyceryl Palmitate | Thickener | 4.50 |
| (and) Glyceryl Stearate (and) Glyceryl | | |
| Ricinoleate (and) Glyceryl Eicosanedioate | | |
| (and) Dextrin Palmitate | | |
| Oryza Sativa (Rice) Bran Extract (and) | Antioxidant | 1.00 |
| Rosmarinus Officinalis (Rosemary) Extract | | |
| (and) Helianthus Annuus (Sunflower) | | |
| Extract (and) Tocopherol | | |
| Butyrospermum Parkii (Shea) Butter (and) | Active | 4.00 |
| Elaeis Guineensis (Palm) Butter (and) | | |
| Cocos Nucifera (Coconut) Oil (and) | | |
| Simmondsia Chinensis (Jojoba) Seed Oil | | |
| (and) Curcuma Longa (Turmeric) Root Extract | | |
| (and) Bisabolol | | |
| Helianthus Annuus (Sunflower) Seed Oil | Emollient | 28.60 |
| Simmondsia Chinensis (Jojoba) Seed Oil | Emollient | 0.10 |
| Rosa Damascena Flower Extract (and) | Fragrance | 0.80 |
| Thuja Occidentalis (White Cedar) Leaf Oil | | |
| (and) Bulnesia Sarmientoi Wood Oil (and) | | |
| Ormenis Multicaulis (Chamomile) Oil (and) | | |
| Eugenia Caryophyllus (Clove) Leaf Oil | | |
| (and) Cymbopogon Martini Oil (and) | | |
| Pelargonium Graveolens Flower (Geranium) | | |
| Oil (and) Fusanus Spicatus Wood (Sandal | | |
| Wood) Oil | | |
| | | 100% |

### Texture Altering

Referring again to FIG. 1, as discussed above, each strand of hair is composed of chemical bonds of hydrogen, salt, and disulfide bonds. The more disulfide that occurs in the hair fiber, the curlier the hair. In one embodiment, the texture altering mixture 106 of the system 100 may minimally break the disulfide bonds of the hair causing the natural curl pattern of the hair shaft to reform, reshape, or loosen into a less coily, curly, wavy, or smoother curl pattern without actually straightening the hair. As a result, the hair will still have the appearance of a version of curls, coils, or waves. It may be more easily managed for care, moisture, and styling, including hair having a tightly coiled or super curly curl pattern. The texture altering mixture 106 is distinguishable from a chemical straightener because although the texture altering mixture 106 modifies by slightly or incompletely breaking disulfide bonds in the hair, the formulation in this mixture is not strong enough and further lacks a fixing agent to break those disulfide bonds enough to straighten the hair. References to “reformation” essentially means loosening, reshaping, or elongating the existing curl pattern of hair, but not to the extent the hair is straightened. The initial or previous curl pattern of hair is preserved while improving manageability. More particularly, the texture altering mixture, and the system itself, does not convert coily, curly, or wavy hair to straight hair as is the result of a chemical straightener. Instead, it essentially stretches the curl, coil, or wave without completely breaking the disulfide bond. Using a heating tool or a fixing agent is not required or necessary for the texture altering mixture to activate on the hair. Warm water may be used during the rinsing portion of the system.

In one embodiment, the texture altering mixture 106 may be substantially a mostly plant-based and/or plant-derived formulation that comprises a small amount or low concentration of sodium hydroxide, for example about 1.25% to 1.75%. The texture altering mixture 106 may be formulated into a light cream (or equivalent) and applied to the hair's root, shaft, and ends for a pre-determined period of time, for example, between about 25 to 60 minutes, depending upon the desired curl pattern. The texture altering mixture 106 may comprise a small amount of sodium hydroxide (NaOH) as a pH adjuster, at least one emulsifier, a humectant, and a hair conditioning component as an active ingredient. The amount of sodium hydroxide present in the texture altering mixture 106 may be increased or decreased, depending upon the desired curl pattern. The preferred range, coupled with the available duration and conditioning agent, is distinguishable from the higher concentration of sodium hydroxide and fixing agent used in a chemical straightener. For example, in one embodiment, 1.35% by weight sodium hydroxide may be used to slightly loosen the curl pattern of the hair, and in another embodiment, 1.48% by weight sodium hydroxide may be used to loosen the curl pattern even further, and so on.

Upon the expiration of the pre-determined time, as discussed in further detail below, the texture altering mixture 106 may be rinsed from the hair with warm water, for example, approximately 32° C. to 43° C., and neutralized using a neutralizing mixture 108 (discussed below). Approximately six to eight weeks after the initial (and each subsequent) application of the texture altering mixture 106, the system 100 may be used to address any new hair growth. The texture altering mixture 106 may be reapplied to the new hair growth and to the entire hair strand if the hair strands have reverted back to the initial or previous curl pattern.

The texture altering mixture 106 is applied to the hair after the porosity mixture and the protective mixture. The duration of time the texture altering mixture 106 is left on the hair may range between about 25-60 minutes, depending on the desired curl pattern. The length of time that the texture altering mixture is on the hair before rinsing it off does not have an adverse effect on the hair and, due to its formulation, does not damage the hair or cause permanent straightening. This formulation coupled with the duration of application is distinguishable from chemical straighteners. Here, use of the texture altering mixture 106 on the hair would not result in the scalp or skin being chemically burned. Also, use of the texture altering mixture 106 would not result in the hair being damaged or suffer from breakage to the extent of changing or eliminating the curl pattern to a straight pattern. Here, the longer the duration coupled with the lower concentration of sodium hydroxide used as a pH adjuster, the more the texture altering mixture 106 has to work on the hair strands to reform, reshape, and loosen the curl pattern, while still maintaining a version of the curl, coil, or wave. By using predominantly plant-based or plant-derived ingredients, the hair strands are not stripped of moisture, weakened, or damaged.

In one embodiment, the texture altering mixture 106 may comprise a mixture of ingredients shown in Table 6 below. All percentages in Table 6 are by weight and approximate for the entire mixture, making up 100% as shown in Table 6.

TABLE 6
Texture Altering Mixture Example

| INCI Name | Purpose | Percentage |
|---|---|---|
| Water | | 65.02 |
| Sodium Hydroxide | pH Adjuster | 1.48 |
| Behentrimonium Chloride | Active-Hair | 0.60 |
| | Conditioning | |
| Polysorbate 60 (and) | Emulsifier | 5.50 |
| Cetearyl Alcohol | | |
| Ceteareth-20 (and) | Emulsifier | 5.20 |
| Cetearyl Alcohol | | |
| C13-15 Alkane | Emollient | 14.00 |
| Simmondsia Chinensis | Emollient | 0.10 |
| (Jojoba) Seed Oil | | |
| Helianthus Annuus | Emollient | 0.10 |
| (Sunflower) Seed Oil | | |
| Propanediol | Humectant | 8.00 |
| | | 100% |

The compositions listed in Table 6 above, namely Behentrimonium Chloride, Polysorbate 60, Ceteareth-20, C13-15 Alkane, and Propanediol are not nonreactive, crosslinking mechanisms or agents in the same or similar manner as the fixing agents in chemical straighteners. Instead, these primarily function as surfactants, emulsifiers, conditioning agents, solvents, and humectants in cosmetic and personal care products. The emulsifiers, emollients, humectant, surfactants, conditioning agents, and solvents in the table above perform temporary, physical, or surface-level actions of conditioning and/or emulsifying rather than chemical crosslinking. Also, lower concentrations of sodium hydroxide as used with the texture altering mixture does not induce physical crosslinking in polymers. In contrast, high concentrations of sodium hydroxide, such as those found in chemical straighteners, can induce physical crosslinking in polymers like polyvinyl alcohol (PVA) by promoting crystallinity.

One or more natural oils are used in the various mixtures of the system, and low levels of sodium hydroxide is used in the texture altering mixture. Testing and observation was conducted concerning scalp sensitivity, with the results reflected in Table 7.

TABLE 7
Scalp and Sensitivity Observations

| Observation | Result |
|---|---|
| Irritation Reports | Low incidence |
| Sensitivity Events | Low incidence |
| Protective Measures | Barrier creams and timing controls effective |

A functional assessment was conducted to determine ingredient performance, particularly, for the texture altering mixture as reflected in Table 8.

TABLE 8
Ingredient Performance

| | | Regulatory-Relevant |
|---|---|---|
| Component | Functional Role | Observation |
| Sodium | Controlled bond | Effective at low |
| | | concentration; |
| Hydroxide (1.35%) | reformation | no rapid fiber collapse |
| | | observed |
| Texture Altering Care | Conditioning & slip | Mitigated dryness and |
| | | friction |
| Creme Base | | |
| Plant-Derived | Lubrication & | Supported post-process |
| Emollients | moisture | manageability |

In one embodiment, the texture altering mixture having about 1.35% by weight sodium hydroxide was compared with a conventional or traditional chemical straighteners concerning the factors for curl pattern outcome, the level of sodium hydroxide, processing control, fiber integrity impact, margin for error, and the intended use. Table 9 reflects the observation:

TABLE 9
Comparison between Texture Altering System and Relaxers

| Metric | Texture Altering System | Traditional Relaxers |
|---|---|---|
| Curl Outcome | Partial loosening | Full straightening |
| Sodium Hydroxide Level | ~1.35% | Typically higher |
| Processing Control | Gradual, visual | Rapid, time-critical |
| Fiber Integrity Impact | Low | Moderate-high |
| Margin for Error | Wider | Narrow |
| Intended Use | Texture modification | Permanent |
| | | straightening |

More particularly, the degree of structural change to the hair strands resulted in partial, graduated modification of the curl loosening rather than permanent breakage of the disulfide bonds leaving a straight hair strand devoid of a curly, coily, or wavy curl pattern. The texture altering mixture 106 uses a low concentration alkaline agent with conditioning counterbalance as the chemical concentration strategy, with the outcome variability of the curl pattern result being adjustable by time and approach. There was no observed cumulative degradation on repeat application of the system when spaced appropriately. Functional performance of the system, and more particularly application of the texture altering mixture, is achieved through low-concentration alkaline agent (about 1.35% by weight NaOH) combined with an integrated conditioning base that moderates fiber stress during processing. In one embodiment, the system is preferably used by professional stylists trained in the system.

### Neutralizing

As a result of applying the texture altering mixture 106 to the hair, the hair's pH level may increase from normal (e.g., 4.5 to 5.5) or neutral (e.g., 7) levels up to 13. The neutralizing shampoo mixture 108 of the system 100 may be used following application of the texture altering mixture 106 to, among other things, gently cleanse the hair and lower the pH of the hair to normal levels and to moisturize the hair. Lowering the hair to normal or neutral pH levels seals the hair cuticle, locks in natural hair oils, prevents unwanted hair breakage, and promotes hair growth.

In addition to returning the hair back to its normal pH level, certain ingredients of the neutralizing shampoo mixture 108 may provide moisture and add smoothness to the hair without the use of silicones, sulfates, or parabens. For example, in one embodiment, as shown in Table 10, guar hydroxypropyltrimonium chloride, aloe barbadensis leaf juice, and the like, may be used to add moisture to the hair.

In one embodiment, the neutralizing shampoo mixture 108 may comprise the ingredients shown in Table 10 below. All percentages in Table 10 are approximate by weight for the entire mixture, making up 100% as shown in Table 10.

TABLE 10
Neutralizing Shampoo Mixture Example

| INCI Name | Purpose | Percentage |
|---|---|---|
| Water | | 59.90 |
| Phenoxyethanol (and) | Preservative | 0.80 |
| Caprylyl Glycol (and) | | |
| Ethylhexylglycerin (and) | | |
| Hexylene Glycol | | |
| Tetrasodium Glutamate | Chelator | 0.20 |
| Diacetate | | |
| Guar Hydroxypropyltrimonium | Active-Hair | 0.25 |
| Chloride | Conditioning | |
| Citric Acid | pH Adjuster | 0.50 |
| Water (and) Sodium | Cleansing | 26.00 |
| Lauroyl Methyl Isethionate | Surfactant | |
| (and) Cocamidopropyl Betaine | | |
| (and) Sodium Cocoyl Isethionate | | |
| (and) Sodium Methyl Oleoyl Taurate | | |
| Cocamidopropyl Betaine | Cleansing | 6.00 |
| | Surfactant | |
| Aloe Barbadensis Leaf Juice | Active | 0.10 |
| Polyquaternium-73 | Active-Hair | 1.60 |
| | Conditioning | |
| Polyquaternium-7 | Active-Hair | 1.20 |
| | Conditioning | |
| Aqua (and) (Wheat Amino Acids | Active-Hair | 0.10 |
| (and) Soy Amino Acids (and) | Conditioning | |
| Arginine HCl (and) Serine (and) | | |
| Threonine | | |
| Sodium Benzoate | Preservative | 0.20 |
| Phenolsulfonphthalein | Color Indicator | 0.002 |
| Caprylyl/Capryl Glucoside (and) | Solubilizer | 1.50 |
| Water (and) Sodium Cocoyl Glutamate | | |
| (and) Glyceryl Caprylate (and) | | |
| Citric Acid (and) Polyglyceryl-6 | | |
| Oleate (and) Sodium Surfactin | | |
| Sorbeth-230 Tetraoleate (and) | Thickener | 0.80 |
| Decyl Glucoside (and) Sorbitan Laurate | | |
| Rosa Damascena Flower Extract (and) | Fragrance | 0.85 |
| Thuja Occidentalis (White Cedar) Leaf | | |
| Oil (and) Eugenia Caryophyllus (Clove) | | |
| Leaf Oil (and) Cymbopogon Martini Oil | | |
| (and) Pelargonium Graveolens Flower | | |
| (Geranium) Oil (and) Fusanus Spicatus | | |
| Wood (Sandal Wood) Oil | | |
| | | 100% |

In one embodiment, the neutralizing shampoo mixture 108 may comprise an indicator that may be activated when residual texture altering mixture 106 is detected in the hair. For example, after rinsing the texture altering mixture 106 from the hair, the neutralizing shampoo mixture 108 may be applied to the hair and worked into a lather. In one embodiment, a color indicator may be activated when residual texture altering mixture 106 is detected in the hair, which may cause the neutralizing shampoo mixture 108 lather to turn a particular color (e.g., pink, etc) to alert a stylist or another that additional shampooing using the neutralizing shampoo mixture 108 is required to remove all of the texture altering mixture 106 from the hair. In one embodiment, as shown in Table 4 above, an indicator may comprise phenolsulfonphthalein; however, other indicators are possible consistent with embodiments of the present disclosure.

When all of the texture altering mixture 106 and any pre-treatment mixture is removed from the hair, the neutralizing shampoo mixture 108 lather may be white or clear. Removing all of the texture altering mixture 106 from the hair may prevent hair breakage or other damage to the hair.

### Moisturizing

Still referring to FIG. 1, the moisturizing shampoo mixture 110 of the system 100 may add moisture back to the hair leaving it hydrated and shiny without the use of silicones, sulfates, or parabens. The moisturizing shampoo mixture 110 may be applied to the hair following application of the neutralizing shampoo mixture 108. The moisturizing shampoo 110 may also remove any residual texture altering mixture 106 and any other pre-treatment mixtures from the hair that was not removed by the neutralizing shampoo mixture 108. In one embodiment, the moisturizing shampoo mixture 110 may include a pH adjuster to assist in returning the hair to normal pH levels following application of the texture altering mixture 106.

In one embodiment, the moisturizing shampoo 110 may comprise the ingredients shown in Table 11 below. All percentages in Table 11 are by weight and approximate for the entire mixture, making up 100% as shown in Table 11.

TABLE 11
Moisturizing Shampoo Mixture Example

| INCI Name | Purpose | Percentage |
|---|---|---|
| Water | | 56.25 |
| Guar | Active-Hair | 0.80 |
| | Conditioning | |
| Hydroxypropyltrimonium Chloride | | |
| Citric Acid | pH Adjuster | 0.05 |
| Cocamidopropyl Hydroxysultaine | Cleansing | 18.00 |
| | Surfactant | |
| Sodium Cocoyl Isethionate | Cleansing | 3.50 |
| | Surfactant | |
| Sodium Lauroyl | Cleansing | 5.00 |
| Methyl Isethionate | Surfactant | |
| Glycol Distearate | Pearlizer | 1.00 |
| Polyquaternium-73 | Active-Hair | 1.20 |
| | Conditioning | |
| Orbignya Oleifera Seed Oil | Emollient | 0.10 |
| Aloe Barbadensis Leaf Juice | Active | 0.10 |
| Phenoxyethanol (and) Caprylyl Glycol | Preservative | 1.00 |
| (and) Ethylhexylglycerin (and) Hexylene | | |
| Glycol | | |
| Water (and) Hydrolyzed Wheat Protein | Active Hair | 1.00 |
| (and) Hydrolyzed Soy Protein (and) | Conditioning | |
| Hydrolyzed Corn Protein | | |
| Decyl Glucoside | Cleansing | 2.50 |
| | Surfactant | |
| Rosa Damascena Flower Extract | Fragrance | 0.90 |
| (and) Bulnesia Sarmientoi Wood Oil | | |
| (and) Ormenis Multicaulis (Chamomile) | | |
| Oil (and) Eugenia Caryophyllus | | |
| (Clove) Leaf Oil (and) Cymbopogon Martini | | |
| Oil (and) Fusanus Spicatus (Sandal Wood) | | |
| Wood Oil Water | | |
| Acrylates Crosspolymer-4 | Thickener | 2.50 |
| Tetrasodium Glutamate | Chelator | 0.20 |
| Sodium Hydroxide | pH Adjuster | 0.40 |
| Citric Acid | pH Adjuster | 0.50 |
| | | 100% |

### Conditioning

Again, referring to FIG. 1, the conditioning mixture 112 of the system 100 may combine protein and moisture to penetrate the hair shaft to both strengthen and moisturize the newly reformed hair. In one embodiment, the conditioning mixture 112 may strengthen the hair using proteins. For example, as shown in Table 6 below, hydrolyzed wheat, hydrolyzed soy and hydrolyzed corn proteins may be used; however, other similar proteins may be used. In one embodiment, the conditioning mixture 112 may add moisture and smoothness to the hair using natural oils rather than silicones, sulfates, or parabens. Such natural oils may include jojoba, avocado, sunflower seed, and bran, as shown in Table 12 below; however, other similar natural oils may be used. Additionally, in one embodiment, the conditioning mixture 112 may have a lightweight formulation that adds bounce and body to the hair. The conditioning mixture may also work together with the neutralizing shampoo mixture 108 and the moisturizing shampoo mixture 110 to further restore, replenish and strength the hair, and may include a pH adjuster to assist the neutralizing shampoo mixture 108 and the moisturizing shampoo mixture 110 in returning the hair to normal pH levels.

In one embodiment, penetration of the conditioning mixture 112 into the hair may be increased by the application of heat. For example, by applying heat to the conditioning mixture 112 while it is on the hair, the conditioning mixture 112 may activate to further fortify and penetrate the hair. Heat may be applied for 30 minutes or less using steam (e.g., via a steamer), a hooded dryer, or the like.

In one embodiment, the conditioning mixture 112 may comprise the ingredients shown in Table 12 below. All percentages in Table 12 are approximate by weight for the entire mixture, making up 100% as shown in Table 12.

TABLE 12
Conditioning Mixture Example

| INCI Name | Purpose | Percentage |
|---|---|---|
| Water | | 77.85 |
| Glycerin | Humectant | 4.00 |
| Tetrasodium Glutamate | Chelator | 0.20 |
| Diacetate | | |
| Guar | Active-Hair | 0.60 |
| | Conditioning | |
| Hydroxypropyltrimonium | | |
| Chloride | | |
| Citric Acid | pH Adjuster | 0.25 |
| Behentrimonium Chloride | Active-Hair | 3.00 |
| | Conditioning | |
| Cetearyl Alcohol | Thickener | 5.50 |
| Simmondsia Chinensis | Emollient | 1.50 |
| (Jojoba) Seed Oil | | |
| Persea Gratissima | Emollient | 1.50 |
| (Avocado) Oil | | |
| Oryza Sativa (Rice) Bran | Antioxidant | 0.10 |
| Extract (and) Rosmarinus Officinalis | | |
| (Rosemary) Extract (and) Helianthus Annuus | | |
| (Sunflower) Extract (and) Tocopherol | | |
| Polyquaternium-37 (and) Dicaprylyl | Active-Hair | 2.50 |
| Carbonate (and) Lauryl Glucoside | Conditioning | |
| Water (and) Hydrolyzed Wheat Protein (and) | Active-Hair | 1.00 |
| Hydrolyzed Soy Protein (and) Hydrolyzed | Conditioning | |
| Corn Protein | | |
| Sodium Benzoate (and) Potassium Sorbate | Preservative | 1.00 |
| (and) Water | | |
| Rosa Damascena Flower Extract | Fragrance | 1.00 |
| (and) Thuja Occidentalis (White | | |
| Cedar) Leaf Oil (and) Ormenis Multicaulis | | |
| (Chamomile) Oil (and) Eugenia Caryophyllus | | |
| (Clove) Leaf Oil (and) Cymbopogon Martini | | |
| Oil (and) Pelargonium Graveolens | | |
| Flower (Geranium) Oil (and) Fusanus | | |
| Spicatus Wood | | |
| (Sandal Wood) Oil | | |
| | | 100% |

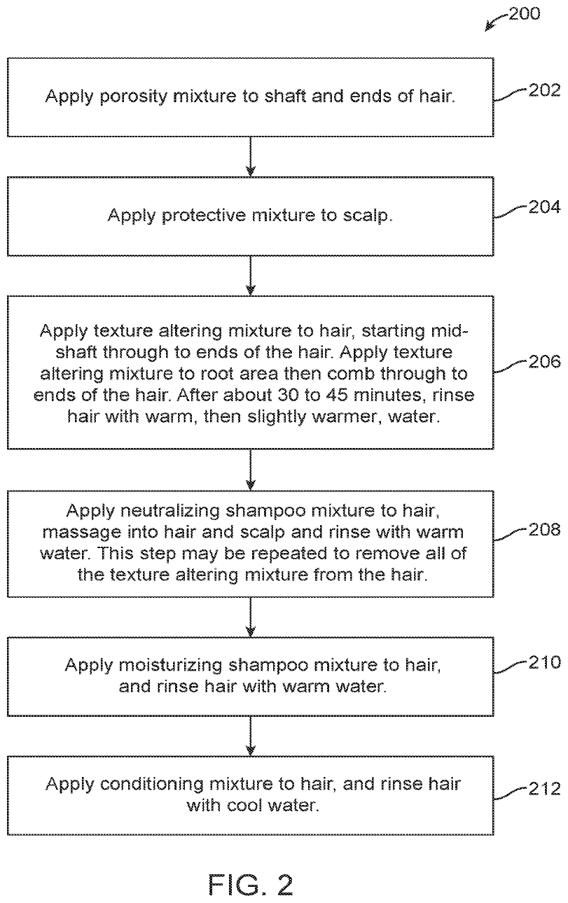

Turning now to FIG. 2, an exemplary method 200 for altering the texture of hair consistent with one or more embodiments of the present disclosure is shown. At step 202, the porosity mixture 102 may be applied to the hair shaft and the ends. As discussed above, the porosity mixture 102 prepares the hair for application of the texture altering mixture 106 by coating the hair with protein and amino acids, or the like, to fill in any pores or open spaces that may exist in the hair cuticle. This coating provides for even distribution and absorption of the texture altering mixture 106, as overly porous hair may absorb the texture altering mixture 106 faster and more unevenly than desired. The porosity mixture 102 may be applied to the hair before applying the texture altering mixture 106.

At step 204, the protective mixture 104 may be applied to the scalp. As discussed above, the protective mixture 104 protects the scalp during application of the texture altering mixture 106 by creating a barrier between the skin and the texture altering mixture 106, thereby reducing the possibility of skin irritation.

At step 206, the texture altering mixture 106 may be applied to the hair starting at the mid-shaft to the ends of the hair. The texture altering mixture may also be applied to the root areas of the hair and combed through to the ends of the hair. After approximately 30 to 45 minutes, depending upon the amount of desired reforming of the curl pattern, the hair may be rinsed with warm water (e.g., approximately 32° C. to 43° C.) and then slightly warmer water to remove the texture altering mixture 106 and the protective mixture 104 from the hair. As previously discussed, without actually straightening the hair, the texture altering mixture 106 non-permanently alters the disulfide bonds of the hair causing the natural curl pattern of the hair shaft to reform and reshape into a less curly, coily, or wavy pattern.

At step 208, the neutralizing shampoo mixture 108 may be massaged into the hair and scalp, and rinsed from the hair with warm water (e.g., approximately 32° C. to 43° C.). This step may be repeated, as needed, to remove all of the texture altering mixture 106 from the hair. In a preferred embodiment, the neutralizing shampoo mixture may be applied twice, rinsing between each application. As discussed above, the neutralizing shampoo mixture 108 may be applied to the hair and massaged into the hair and scalp to gently cleanse the hair, to add moisture to the hair, and to lower the pH of the hair to normal levels. As further discussed above, the neutralizing shampoo mixture 108 may include an indicator (e.g., color indicator, or the like) that lets a stylist or another know when the texture altering mixture 106 is completely removed from the hair. For example, after rinsing the texture altering mixture 106 from the hair, the neutralizing shampoo mixture 108 may be applied to the hair and worked into a lather. In one embodiment, a color indicator may be activated when residual texture altering mixture 106 is detected in the hair, which may cause the neutralizing shampoo mixture 108 lather to turn a particular color (e.g., pink, etc).

At step 210, the moisturizing shampoo mixture 110 may be applied to the hair, as discussed above, to add moisture back to the hair, leaving it hydrated and shiny without the use of silicones, sulfates, or parabens. The moisturizing shampoo mixture 110 may then be rinsed from the hair using warm water. In a preferred embodiment, the moisturizing shampoo mixture is applied at least one time then rinsed.

At step 212, as discussed above, the conditioning mixture 112 combines protein and moisture, using natural oils rather than silicones, sulfates, or parabens, to penetrate the hair shaft to both strengthen and moisturize the newly reformed hair. Once applied to the hair, the conditioning mixture 112 may be rinsed from the hair with cool water to seal the hair cuticle. In one embodiment, as discussed above, heat may be applied to the hair for 30 minutes or less, which may cause the conditioning mixture 112 to further penetrate the hair shaft to fortify, strength, and moisturize the hair.

In another embodiment, the method of using the system includes the steps of: applying the porosity mixture to the hair as described above; applying the protective mixture to at least the scalp and roots of the hair; applying the texture altering mixture after the porosity mixture and the protective mixture, wherein for an initial application the texture altering mixture is applied to the entire hair strand from root to shaft to the end of the hair. The hair does not revert back to the original or previous curl or coil over time; however, the texture altering mixture 106 can be applied on subsequent occasions to further loosen or reform the modified curl pattern of the hair shaft without the curl pattern ever fully straightening. On subsequent occasions, the texture altering mixture may be applied to the processed hair to retain the modified curl pattern or only to the new growth. The texture altering mixture may be selectively applied to the hair and may remain for a selective time frame without causing damage to the hair or overprocessing, with a selective time frame ranging from about 25 to 60 minutes. In a preferred embodiment, the texture altering mixture may remain on the hair for about 45 minutes as it activates and works to stretch the curl pattern of the hair strands, while still maintaining some of the coils, curls, or waves of the curl pattern. It should be noted that the decreased amount of sodium hydroxide is a significant distinction among the invention, a relaxer, and a texturizer. A goal of the present invention is to conform a curl patter of hair into a stretched, reformed, redefined, reshaped, lengthened, or loosened curly, coily, or wavy curl pattern of hair while simultaneously keeping the treated hair conditioned and moisturized. The result still resembles a natural curl pattern of hair, unique to the individual's particular curl patterns. This versatility in the result of the curls, coils, and waves in the hair make the system beneficial to naturals desiring a modified version of their curly/coily natural hairstyle. In contrast, the result of a chemical straightener, for example a relaxer, is the same on nearly any curl pattern of hair-taking a curly, coily, or wavy hair texture, permanently breaking the keratin disulfide bonds, and making it straight hair.

Table 13 notes the difference in concentration of sodium hydroxide when formulated for application on the hair to alter a curl pattern. For example, 0.1% by weight sodium hydroxide in a mixture would have little to no effect on changing a curl pattern of hair. 1.35% by weight sodium hydroxide in a mixture would effectively alter the texture or curl pattern of hair to begin reshaping it, but at this concentration in the described combination of ingredients, the result would not be straight hair. The mixture composition having 1.35% by weight sodium hydroxide is preferred. With 1.55% by weight sodium hydroxide in a mixture, the curl pattern would experience more elongation or reformation of the hair, but at this concentration in the described combination of ingredients, the result would not be straight hair. At these sodium hydroxide concentrations, the hair is capable of reverting back to its initial or previous curl pattern of coils, curls, or waves. At about 1.6% or more by weight sodium hydroxide in a mixture, the sodium hydroxide would effectively and critically break the hair's disulfide bonds to the point of losing any curl, coil, or wavy, resulting in straight hair.

In another embodiment, the conditioning mixture comprises one or more proteins to strengthen the hair; wherein at least one of the one or more proteins is selected from the group consisting of hydrolyzed wheat protein, hydrolyzed soy protein, or hydrolyzed corn protein; and wherein the conditioning mixture is configured to improve the condition of the hair.

The recited concentration amounts, percentages, and selection of ingredients, at least as identified in the embodiments, are critical iterations of those combinations of ingredients. The tested ingredients, concentrations, and percentages yield among the better results for use and implementation of the disclosed systems and methods. The recited genus and species may be used in the composition. Alternatively, the composition may use the recited general term for the associated genus and species. Furthermore, the sequence of steps for applying each mixture of the system is essential to producing the requisite results.

All references throughout this application, for example, patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application. For example, a reference that is partially inconsistent is incorporated by reference except for the partially inconsistent portion of the reference.

Portions of the disclosure of this patent document may contain material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Any element in a claim that does not explicitly state “means for” performing a specified function, or “step for” performing a specified function, is not to be interpreted as a “means” or “step” clause as specified in 35 U.S.C. § 112 16. In particular, any use of “step of” in the claims is not intended to invoke the provision of 35 U.S.C. § 112 16.

Persons of ordinary skill in the art may appreciate that numerous configurations may be possible to enjoy the functional benefits of the inventive systems and methods. Thus, given the wide variety of configurations and arrangements of embodiments of the present disclosure the scope of the invention is reflected by the breadth of the claims below rather than narrowed by the embodiments described above.

I claim:

1. A hair texture altering system, the hair texture altering system comprising:
a. a texture altering mixture comprising a low concentration alkaline agent and an integrated conditioning agent for keratin disulfide bond loosening and reforming a curl pattern of hair for graduated curl pattern modification while preserving curl structure for subsequent application for further loosening and reforming the curl pattern and without straightening the hair;
b. a porosity mixture, wherein the porosity mixture is formulated to prepare the hair for receiving the texture altering mixture;
c. a protective mixture, wherein the protective mixture is formulated to form a barrier between the texture altering mixture and a scalp portion of a head located at a root of the hair;
d. a neutralizing shampoo mixture, wherein the neutralizing shampoo mixture is formulated to return the hair to its normal pH level and to add moisture to the hair;
e. a moisturizing shampoo mixture, wherein the moisturizing shampoo mixture is formulated to moisturize the hair, wherein improving a condition of the hair comprises moisturizing the hair using one or more natural oils; and
f. a conditioning mixture, wherein the conditioning mixture is configured to improve the condition of the hair.

2. The hair texture altering system of claim 1, wherein the curl pattern of hair is selected from the group consisting of curly, super curly, coily, or wavy.

3. The hair texture altering system of claim 2, wherein the integrated conditioning agent of the texture altering mixture comprises one or more natural oils to add moisture to the hair.

4. The hair texture altering system of claim 3, wherein one of the one or more natural oils comprises about 0.10 by weight jojoba seed oil.

5. The hair texture altering system of claim 3, wherein one of the one or more natural oils comprises about 0.10 by weight sunflower seed oil.

6. The hair texture altering system of claim 1, wherein texture altering mixture is formulated to remain on the hair between about 25 to 60 minutes before rinsing with water.

7. The hair texture altering system of claim 1, wherein each of the texture altering mixture, the protective mixture, the neutralizing shampoo mixture, and the conditioning mixture do not include parabens, sulfates, silicones, synthetic fragrance, mineral oil, petrolatum, or synthetic petrochemicals.

8. The hair texture altering system of claim 1, wherein the low concentration alkaline agent of the texture altering mixture comprises about 1.35% by weight sodium hydroxide.

9. The hair texture altering system of claim 1, wherein the low concentration alkaline agent of the texture altering mixture comprises about 1.48% by weight sodium hydroxide.

10. The hair texture altering system of claim 1, wherein the protective mixture comprises one or more natural oils and a base, wherein the base comprises at least one of the one or more natural oils.

11. The hair texture altering system of claim 10, wherein the at least one of the one or more natural oils of the base of the protective mixture comprises about 55.0% by weight sunflower seed oil.

12. The hair texture altering system of claim 1, wherein the protective mixture comprises turmeric root extract.

13. The hair texture altering system of claim 1, wherein the neutralizing shampoo mixture comprises about 0.10% by weight of aloe barbadensis leaf juice to add moisture to the hair.

14. The hair texture altering system of claim 1, wherein the moisturizing shampoo mixture comprises about 0.10% by weight aloe barbadensis leaf juice to add moisture to the hair.

15. The hair texture altering system of claim 1, wherein at least one of the one or more natural oils for the moisturizing shampoo mixture is selected from the group consisting of avocado oil, guar hydroxypropyltrimonium chloride, and bran extract.

16. The hair texture altering system of claim 1, wherein the conditioning mixture comprises about 1.50% by weight jojoba seed oil.

17. The hair texture altering system of claim 1, wherein the conditioning mixture comprises about 1.50% by weight avocado oil.

18. A hair texture altering system of claim 1, wherein the conditioning mixture comprises one or more proteins to strengthen the hair, and wherein at least one of the one or more proteins is selected from the group consisting of hydrolyzed wheat protein, hydrolyzed soy protein, or hydrolyzed corn protein.

19. A conditioning mixture comprising:
a. one or more proteins to strengthen the hair;
b, wherein at least one of the one or more proteins is selected from the group consisting of hydrolyzed wheat protein, hydrolyzed soy protein, or hydrolyzed corn protein; and
c. wherein the conditioning mixture is configured to improve the condition of the hair.

20. The conditioning mixture of claim 19, wherein the conditioning mixture comprises about 1.50% by weight jojoba seed oil.

21. The conditioning mixture of claim 19, wherein the conditioning mixture comprises about 1.50% by weight avocado oil.

22. A method of altering hair texture, the method comprising the steps of:
a. applying a porosity mixture to the hair, wherein the porosity mixture is formulated to prepare the hair for application of a texture altering mixture;
b. applying a protective mixture to at least the scalp portion of a head located at the root of the hair after application of the porosity mixture, wherein the protective mixture is formulated to create a barrier between a texture altering mixture and the scalp portion of the head located at the root of the hair;
c. applying the texture altering mixture after applying the porosity mixture and the protective mixture, wherein the texture altering mixture comprises a low concentration alkaline agent and an integrated conditioning agent of one or more natural oils for keratin disulfide bond loosening and reforming a curl pattern of hair for graduated curl pattern modification while preserving curl structure for subsequent application for further loosening and reforming the curl pattern and without straightening the hair, and wherein the texture altering mixture is free from parabens, sulfates, silicones, synthetic fragrance, mineral oil, and petrolatum;
d. applying a neutralizing shampoo mixture to the hair after the texture altering mixture is rinsed from the hair as a first application of the neutralizing shampoo mixture, then rinsing the first application of neutralizing shampoo mixture from the hair, then applying at least a second application of the neutralizing shampoo mixture to the hair, wherein the neutralizing shampoo mixture is formulated to return the hair to its normal pH level and to add moisture to the hair;
e. applying a moisturizing shampoo mixture to the hair after the at least second application of the neutralizing shampoo mixture is rinsed from the hair, wherein the moisturizing shampoo mixture is formulated to further moisturize the hair and to remove any residual of the texture altering mixture, the protective mixture, and the porosity mixture from the hair; and
f. applying a conditioning mixture to the hair after the moisturizing shampoo is rinsed from the hair, wherein the conditioning mixture is configured to improve the condition of the hair, and wherein improving the condition of the hair comprises moisturizing and strengthening the hair.

23. The method of altering hair texture of claim 22, wherein the low concentration alkaline agent of the texture altering mixture comprises about 1.35% by weight sodium hydroxide.

24. The method of altering hair texture of claim 22, wherein the low concentration alkaline agent of the texture altering mixture comprises about 1.48% by weight sodium hydroxide.
